(19) United States
(12) Patent Application Publication
(10) Pub. No.: US 2019/0054093 A1
(43) Pub. Date: Feb. 21, 2019

(54) TREATMENT OF SARCOPENIA WITH ECDYSTEROIDS

(71) Applicant: Nektium Pharma SL, Las Palmas (ES)

(72) Inventors: Miguel Jimenez del RIO, Telde (ES); Jose Maria ZUBEL-DIA FERNANDEZ, Las Palmas (ES); Aaron HERNANDEZ SANTANA, Las Palmas (ES); Julia Charlotte WIEBE, Las Palmas (ES)

(21) Appl. No.: 15/780,527

(22) PCT Filed: Apr. 2, 2015
(86) PCT No.: PCT/IB2015/052453

§ 371 Date: May 31, 2018

Related U.S. Application Data
(60) Provisional application No. 61/975,212, filed on Apr. 4, 2014.

(51) Int. Cl. A61K 31/575; A61K 36/21; A61K 36/53; A61K 36/28; A61K 36/88; A61K 31/7048; A61K 36/9066; A61K 31/12; A61K 36/67; A61K 31/357; A61K 31/353; A61K 36/16; A61K 36/9068; A61K 36/258; A61K 31/05; A61K 36/33; A61K 36/8945; A61K 36/752; A61K 31/565; A61P 21/00

(52) U.S. Cl. CPC A61K 31/575; A61P 21/00; A61K 36/53; A61K 36/28; A61K 36/88; A61K 31/7048; A61K 36/9066; A61K 31/12; A61K 36/67; A61K 31/357; A61K 31/353; A61K 36/16; A61K 36/9068; A61K 36/258; A61K 31/05; A61K 36/33; A61K 36/8945; A61K 36/752; A61K 31/565; A61K 36/21

(57) ABSTRACT

The current disclosure relates to methods for treating loss of muscle mass, by administering compositions comprising a combination of: 20-hydroxy-ecdysone (20HE) or a pharmaceutically acceptable salt thereof; and at least one ecdysteroid selected from the group consisting of polypodine, makisterone A, integristerone, taxisterone, lesterone, rapisterone, inokonesterone, carthamosterone, rubrosterone, leuzeasterone, ayugasterone, turkestrone, salts thereof, and derivatives thereof. The composition optionally further comprises an effective amount of hesperidin.

### FIELD

This disclosure relates to a composition for slowing, preventing and/or treating the loss of muscle tissue in mammalian subjects. This disclosure further relates to a composition without side effects of common pharmacological drugs used in treating the loss of muscle mass and strength. The disclosure refers to a mixture of bio-available ecdysteroids obtained from plants, including but not limited to Ajuga turkestanica, Rhaponticum carthamoides, Cyanotis longfolia, and Cyanotis arachnoidea.

### BACKGROUND

The potential for any substance to increase protein synthesis in muscle by-passing secondary effects common to synthetic steroid drugs is an attractive approach for managing sarcopenia. Sarcopenia is a condition in which subjects have progressive generalized loss of skeletal muscle mass and function. Skeletal muscle tissue accounts for almost half of the human body mass. Its contractions power human body movements and are essential to maintaining stability. Muscle tissue is also heavily involved in glucose homeostasis. Given these important roles in humans, any deterioration in the contractile, material, and metabolic properties of skeletal muscle has extremely important effects on health.

The term “sarcopenia” describes the loss of muscle tissue. Sarcopenia typically occurs during aging, but it may be a symptom of other conditions, such as obesity, advanced cancer, chronic kidney and heart failure, and AIDS. The term is also commonly used to describe its clinical manifestation. Changes during aging relate to the mass, composition, contractile properties, and material properties of muscle tissue, as well as the function of tendons. Morphologically there are losses in both slow and fast motor units coupled with fiber atrophy. These losses results in decreased muscle power (i.e. rising form a chair or climbing steps), and put more pressure in those surviving remaining fibers as they become overworked. Additionally, muscle tissue becomes infiltrated by lipids, either contained by adipocytes or directly deposited inside the muscle fibers. Functionally correct motor neurons are also necessary for the survival of muscle fibers. Age-related changes have been noted in the neuromuscular junction, with reduced number but increased size of terminal areas and reductions in the number of synaptic vesicles.

Aging is associated with decreased expression of humoral factors that promote protein synthesis, and increased endocrine and inflammatory factors that affect negatively protein balance.

Age-related loss of skeletal muscle contractile power is one of the clinical consequences most commonly linked with sarcopenia. The decline in muscle power has been established in both genders, under multiple loading conditions, in multiple limbs, and in both cross-sectional and longitudinal studies. The most important anatomic sites for muscle function measurement have been primarily in the lower body. These muscles are critical for daily function. Power and strength losses in the lower limbs carry the largest risk factors for falls and other sources of injury and disability. Imaging studies also show loss of mass with age. For example, leg lean mass decreases approximately by 1% per year.

Prospective cohort studies as well as observational studies have demonstrated a clear association between sarcopenia and adverse clinical outcome, especially in the older population:
Measures of lower-body weakness have been correlated to incidence of any fall with odd ratios ranging from 1.2 to 2.5, to injurious falls with odd ratios around 1.5, and to recurrent falls with much higher odd ratios, ranging from 2.2 to 9.9. Upper body weakness is also correlated to fall incidence (odd ratios for incident falls from 1.2 to 2.3, odd ratios of 1.4-1.7 for recurrent falls). Lower extremity weakness is a better predictor of falls though.
Reduced strength of the hip and other lower leg muscles, in addition to impaired neuromuscular activation, may be implicated in poor recovery from falls. In addition to falls, muscle weakness and reduced muscle mass have been associated with incident disability
Thigh muscle cross-sectional area and knee extension torque have also been shown to correlate to incident hip fracture. For example, Lang et al observed that knee extension torque and low cross-sectional area individually resulted in increased risk of incident hip fracture by 50-60%, independent of bone mineral density.
Loss of mobility and injuries resulting for ever decreasing loss of muscle mass and power also establish a positive feedback, vicious circle. Functional losses induced by sarcopenia increase the difficulties associated with procuring adequate nutrition and increase the effort required to undertake exercise. This combination of nutritional loss and reduced physical activity levels results in further loss of muscle mass and power, exacerbating the process of sarcopenia. The resulting decrements, if left uncorrected lead to a loss of independence which may or may not be preceded by injury or illness.
As previously stated, the muscle is a key player in glucose homeostasis. Loosing muscle mass is also associated with insulin resistance in both non-obese and obese individuals and abnormal blood glucose levels in obese individuals, especially in those younger than 60 years of age.
Sarcopenia is a difficult health problem without an ideal therapeutic option. Although treating the cause of a disease is preferred, most of the conditions above described are difficult to treat (i.e. aging, advanced cancer, or chronic kidney and heart failure). Other strategies have looked at avoiding the loss of muscle:
Exercise increases cardiovascular fitness and endurance, mitochondrial volume and activity, muscle mass and strength, skeletal muscle protein synthesis and muscle fiber size. However, it requires motivation that may remain low amongst individuals already sarcopenic. Also, some people will not be able to engage in these types of programs due to their baseline pathology (i.e. bed ridden cancer patients).
Nutritional supplementation with extra doses of proteins have shown varying degrees of success in increasing muscle mass and strength, but it may reduce natural food intake. Also, patients with chronic renal failure need to reduce protein intake to avoid building up dangerous levels of ammonia in the blood.
Hormone therapy includes the use of testosterone, estrogen and/or growth hormone. There is poor evidence of increased muscle mass and unconvincing evidence of increased gains in strength. Side effects include masculinization in females, increased risk of prostate or breast cancer, fluid retention, and orthostatic hypotension to name a few.
Although Vitamin D supplementation has shown it could potentially reduce the number of fractures from falls, the evidence for increased muscle strength is variable at best. Also, it requires monitoring of renal function.
Some investigators have proposed angiotensin-converting enzyme (ACE) inhibitors with variable results as well. This therapy also carries the risk of nephritis.

Ecdysteroids are polyhydroxylated ketosteroids with long carbon side chains. These steroid hormones control moulting and reproduction in arthropods, but their role in plants is less well known as they do not elicit any of the classical plant hormone responses. Plants may use ecdysteroids as a chemical defense against insects by disrupting their hormonal balance and molting process. Ecdysteroids are structurally different from mammalian steroids, and they are not expected to bind to vertebrate steroid receptors. However, anabolic effects have been reported in vertebrates: increased growth in mice, rats, sheep, or pigs, and increased physical performance without training in rats with increased synthesis of myofibrillar proteins.

The discovery of these steroid molecules in 1966 in several plant species led to their availability in large amounts for pharmacologic studies in search of safer, more specific insecticides. While showing no signs of toxicity, ecdysteroids had other possible beneficial effects that could support their use in folk medicine such as immunomodulation, antiarrythmic, hepatoprotective, or antidiabetes effects.

Although there are many plants known to contain significant amounts of these bioactive compounds we will refer to three examples as described below:

1. The genus Ajuga (Labiatae) is comprised of more than 40 species widely distributed in temperate regions of both hemispheres and contains at least three classes of potentially bioactive compounds: clerodane diterpenes, phytoecdysteroids and iridoid glycosides. Ajuga turkestanica (Regel) Briq. is a perennial herb growing mainly in Central Asia known as a rich source of bioactive substances and used by local people to treat heart diseases, muscle and stomach aches. With regards to phytoecdysteroids several bioactive compounds have been isolated including turkesterone, 20-hydroxyecdysone (20HE), cyasterone, cyasterone 22-acetate, ajugalactone, ajugasterone B, α-ecdysone and ecdysone 2, 3-monoacetonide. A characteristic feature of Ajuga turkestanica is the presence of the C11-hydroxylated turkesterone, which has not been observed in other species of the same genus.
2. Rhaponticum carthamoides (Willd.) Iljin, also known as maral root or Russian leuzea, is a perennial herb that belongs to the family Ateraceae. This herb is endemic in the Altai and Saian mountains of South Siberia and grows naturally in the alpine and subalpine meadows at 1200-2300 m above sea level. Several classes of bioactive compounds have been isolated from various parts of the plant. These include organic acids, flavonoids and ecdysteroids. With regards to the latter, 20HE is the most abundant in various parts with concentrations ranging from 0.04 to 1.51%. Other similar compounds include ecdysterone, inokosterone and other ones adding up to at least 50 different phytosteroids.
3. Cyanotis (syn. Tonningia) is a genus of mainly perennial plants in the family Commelinaceae with an estimated 50-100 species disseminated around the world, one of which is Cyanotis arachnoidea (C. B. Clarke). Investigators have isolated beta-ecdysone, ajugasterone, cyanosterone, and 20HE amongst other phytoecdysteroids.

Various embodiments disclosed herein relate to use of ecdysteroids in sports medicine. Ecdysteroids are useful in sports medicine due to their function as an anabolic composition. Muscle growth may be stimulated by administering a formulation compriksing a combination of 20-hydroxyecdysone (20HE) and at least one additional ecdysteroid. The additional ecdysteroid may be selected from the group consisting of polypodine, makisterone A, integristerone, taxisterone, lesterone, rapisterone, inokonesterone, carthamosterone, rubrosterone, leuzeasterone, ayugasterone, turkestrone, salts thereof, derivatives thereof, and mixtures thereof. Alternatively, muscle growth may be stimulated by administering a formulation comprising a combination of 20HE and hesperidin. Also, muscle growth may be stimulated by administering a formulation comprising a combination of 20HE, at least one additional ecdysteroid, and hesperidin.

These examples are the subject of our own investigation which is described later. They are herbal plants with different origins from which bioactive ecdysteroids can be isolated and obtained by (industrially scalable) chemical extraction. Again, the discovery of ecdysteroids in plants has allowed the possibility of their industrial applications. A comprehensive compilation of natural occurring phytosteroidal compounds is attached in appendix A.

### SUMMARY

The incidence and prevalence of sarcopenia are expected to increase as a higher percentage of the population ages and the life-expectancy increases in the elderly. Various exemplary embodiments of the plant-derived ecdysteroid-rich extract provide a safe and effective method in preventing, slowing or treating sarcopenia.

Various exemplary embodiments disclosed herein relate to methods of treating loss of muscle mass in a patient in need thereof, by administering to said patient an effective amount of a composition comprising a combination of 20-hydroxyecdysone (20HE) or a pharmaceutically acceptable salt thereof; and at least one ecdysteroid selected from the group consisting of polypodine B, makisterone A, integristerone, taxisterone, lesterone, rapisterone, inokonesterone, carthamosterone, rubrosterone, leuzeasterone, ayugasterone, turkestrone, salts thereof, and derivatives thereof.

In various embodiments disclosed herein, methods of treating loss of muscle mass in a patient in need thereof involve administering to said patient an effective amount of a composition comprising an extract of at least one plant selected from the group consisting of spinach, Ajuga turkestanica, Rhaponticum carthamoides, and Cyanotis arachnoidea. In various embodiments, the extract of spinach, Ajuga turkestanica, Rhaponticum carthamoides, and/or Cyanotis arachnoidea comprises 20HE or a pharmaceutically acceptable salt thereof; and at least one ecdysteroid selected from the group consisting of polypodine B, makisterone A, integristerone, taxisterone, lesterone, rapisterone, inokonesterone, carthamosterone, rubrosterone, leuzeasterone, ayugasterone, turkestrone, salts thereof, and derivatives thereof.

In various exemplary embodiments, loss of muscle mass in a patient is treated by administering to the patient an effective amount of a composition comprising 20HE and at least one ecdysteroid in a ratio of 1 part 20HE to between 0.1 and 50 parts of said at least one ecdysteroid.

In various exemplary embodiments, loss of muscle mass in a patient is treated by administering to the patient an effective amount of a composition comprising 20HE and at least one ecdysteroid, where the composition further comprises an effective amount of hesperidin.

In various exemplary embodiments, loss of muscle mass in a patient is treated by administering to the patient an effective amount of a composition comprising 20HE and at least one ecdysteroid, where the composition further comprises an effective amount of hesperidin. The composition may comprise from about 200 to about 5 parts by weight of hesperidin and about one part by weight of the combination of 20HE and at least one ecdysteroid; from about 100 to about 10 parts by weight of hesperidin and about one part by weight of the combination of 20HE and at least one ecdysteroid; or from about 50 to about 15 parts by weight of hesperidin and about one part by weight of the combination of 20HE and at least one ecdysteroid. In various embodiments, the composition comprises about 20 parts by weight of hesperidin and about one part by weight of the combination of 20HE and at least one ecdysteroid.

In various exemplary embodiments, loss of muscle mass in a patient is treated by administering to the patient an effective amount of a composition comprising an effective amount of:

a) a composition comprising a combination of:
20-hydroxy-ecdysone (20HE) or a pharmaceutically acceptable salt thereof; and
at least one ecdysteroid selected from the group consisting of polypodine, makisterone A, integristerone, taxisterone, lesterone, rapisterone, inokonesterone, carthamosterone, rubrosterone, leuzeasterone, ayugasterone, turkestrone, salts thereof, and derivatives thereof; and
   b) hesperidin.

Various exemplary embodiments relate to use of a mixture of ecdysteroids naturally present in plants suitable for harvesting for preventing, slowing or treating the loss of muscle mass and strength by down-regulating the expression of molecular pathways that break down the muscle. The mixture comprises an effective amount of 20-hydroxy-ecdysone (20HE) or a pharmaceutically acceptable salt of 20HE; an effective amounts of at least one biochemically active ecdysteroid compound. Ecdysteroid compounds are plant-derived terpenoid derivatives of triterpene molecules. More specifically, ecdysteroids are plant-derived, polyhydroxylated ketosteroids with long carbon side chains. Suitable ecdysteroids for use in the compositions disclosed herein include, but are not limited to, polypodine, makisterone A, integristerone, taxisterone, lesterone, rapisterone, inokonesterone, carthamosterone, rubrosterone, leuzeasterone, ayugasterone, turkestrone, or their derivatives.

These active components are obtained by an extraction process which is suitable for industrial up scaling and yields a number of related ecdysteroids in the final product in variable amounts, but pharmacologically active and analytically detectable in the final mixture

Various exemplary embodiments relate to use of a mixture of ecdysteroids obtained from plants in the manufacture of a medicament for slowing the breakdown of muscle proteins induced by immobilization, aging, chronic disease, and/or chronic infection.

Various exemplary embodiments relate to use of a mixture of ecdysteroids obtained from plants in the manufacture of a medicament for improving the protein content, mass and/or strength of skeletal muscle in mammals at risk of or suffering from sarcopenia.

Various exemplary embodiments relate to the use a mixture of ecdysteroids obtained from plants in the manufacture of a medicament for shutting down cell mechanisms that break down proteins in skeletal muscles and lead to a loss of muscle mass and/or muscle strength. These cell mechanisms are typically stimulated in situations where sarcopenia develops.

Various exemplary embodiments relate to use a mixture of ecdysteroids obtained from plants mixed with hesperidin in the manufacture of a medicament for improving the protein content, mass and/or strength of skeletal muscle in mammals at risk of or suffering from sarcopenia.

The plant extract (or a mixture of different plant extracts) can be administered orally, topically, rectally or parenterally, although orally is preferred. The plant extract (or a mixture of different plant extracts) is applicable to humans, pet animals and industrial animals.

In various embodiments, loss of muscle mass in a patient is treated by administering to the patient an effective amount of a composition comprising 20HE and at least one ecdysteroid, in combination with an effective amount of an extract of Curcuma longa, comprising curcumin, demethoxycurcumin, bis-demethoxycurcumin. The extract of Curcuma longa is effective to promote muscle regeneration. The combination of 20HE and at least one ecdysteroid may comprise 10%, 20%, 30%, 40%, 50%, 60%, 70%, 80%, or 90% by weight of the composition, based on the combined weight of the extract of Curcuma longa and the 20HE/ecdysteroid composition. In various embodiments, the 20HE/ecdysteroid composition may comprise from 10% to 90%, from 30% to 70%, or from 40% to 60% by weight of the composition, based on the combined weight of the extract of Curcuma longa and the 20HE/ecdysteroid composition.

The 20HE/ecdysteroid/Curcuma longa extract may further comprise from 5% to 50%, from 10% to 40%, or from 15% to 30% by weight of an effective amount of an additional component effective to inhibit curcumin glucuronidation. This additional component may be an extract of Piper nigrum; an extract of Cardo mariano; an extract of Allium cepa; silibinin; or quercetin.

In various embodiments, loss of muscle mass in a patient is treated by administering to the patient an effective amount of a composition comprising 20HE and at least one ecdysteroid, in combination with an effective amount of a bioactive component selected from the group consisting of:
an extract of Ginkgo biloba in an amount effective to increase muscle mass, or delay sarcopenia;
an extract of Zingiber officinale comprising 6-gingerol, 6-shogaol, 6-paradol, or a mixture thereof, said extract of Zingiber officinale being present in an amount effective to promote recovery of muscle strength following exercise;
an extract of ginseng in an amount effective to delay sarcopenia;
resveratrol in an amount effective to stimulate at least one of i) activation of satellite cells, ii) myogenesis in myscle tissue; and iii) hypertrophy in muscle tissue;
an extract of prickly pear cactus comprising at least one phyto-estrogen;
an extract of Dioscorea napponica comprising dioscin, said extract of Dioscorea napponica being present in an amount effective to promote at least one of osteoblast differentiation and osteoclast inhibition;
a secosteroid hormone;
an extract of Citrus sinensis in an amount effective to stimulate at least one of i) activation of satellite cells, ii) myoblast differentiation; and iii) osteoblast differentiation:

Loss of muscle mass in a patient may be treated by administering to the patient an effective amount of a composition comprising 20HE and at least one ecdysteroid, in combination with an effective amount of a bioactive component selected from the group consisting of:
from 10% to 90%, from 20% to 70% or from 30% to 50% of an extract of Ginkgo biloba;
from 10% to 90%, from 20% to 70% or from 30% to 50% of an extract of Zingiber officinale comprising 6-gingerol, 6-shogaol, 6-paradol, or a mixture thereof, said extract of Zingiber officinale being present in an amount effective to promote recovery of muscle strength following exercise;
from 10% to 90%, from 20% to 70% or from 30% to 50% of an extract of ginseng in an amount effective to delay sarcopenia;
from 10% to 90%, from 20% to 70% or from 30% to 50% of resveratrol in an amount effective to stimulate at least one of i) activation of satellite cells, ii) myogenesis in myscle tissue; and iii) hypertrophy in muscle tissue;
from 10% to 90%, from 20% to 70% or from 30% to 50% of an extract of prickly pear cactus comprising at least one phyto-estrogen;
from 10% to 90%, from 20% to 70% or from 30% to 50% of an extract of Dioscorea napponica comprising dioscin, said extract of Dioscorea napponica being present in an amount effective to promote at least one of osteoblast differentiation and osteoclast inhibition;
from 10% to 90%, from 20% to 70% or from 30% to 50% of a secosteroid hormone;
from 10% to 90%, from 20% to 70% or from 30% to 50% of an extract of Citrus sinensis in an amount effective to stimulate at least one of i) activation of satellite cells, ii) myoblast differentiation; and iii) osteoblast differentiation, where all percentages are based on the combined weight of 20HE, the ecdysteroid(s), and the bioactive component.

### BRIEF DESCRIPTION OF THE DRAWINGS

For a fuller understanding of the nature and desired objects of the present application, reference is made to the following detailed description taken in conjunction with the accompanying figures wherein:

FIG. 1 is UPLC chromatograms of (A) Ajuga turkestanica whole plant and (B) the corresponding freeze-dried powder extract measured at 245 nm, with the chemical structures of the two most abundant ecdysteroids.

FIG. 2 shows the effect of Ajuga turkestanica extract and methandrostenolone on Caspase-3 (Casp3) and myostatin (Mstn) gene expression levels in C2C12 muscle cells. Test groups were treated with 20 ppm of ATE for 6 hours (n=3).

FIG. 3 shows the relative expression of Myomesin 1 induced by Ajuga turkestanica extract at different dose levels in myotubes, compared with a control group (C2C12 cells with vehicle) at 3 different time-points (3, 6 and 24 h).

FIG. 4 shows the relative expression of Caspase 3 when myotubes cultures were exposed to ascending doses of Rhaponticum carthamoides and Cyanotis arachnoidea, using 20HE and turkesterone standards as positive controls. CASP3: Caspase 3. C: control. 20HE: 20 Hydroxy-ecdysone. RCE: Rhaponitcum carthamoides extract. TURK: Turkesterone. CAE: Cyanotis arachnoidea extract. PPM: Parts per million.

FIG. 5 shows the relative expression of Myostatin when myotubes cultures were exposed to ascending doses of Rhaponticum carthamoides and Cyanotis arachnoidea, using 20HE and turkesterone standards as positive controls. MSTN: myostatin. C: control. 20HE: 20 Hydroxy-ecdysone. RCE: Rhaponitcum carthamoides extract. TURK: Turkesterone. CAE: Cyanotis arachnoidea extract. PPM: Parts per million.

FIG. 6 shows the increased protein synthesis measured by incorporation of radiolabeled leucine (³H-Leu) with different concentrations of Rhaponticum carthamoides vs. a control group. DPM: decays per minute. TP: total protein. Rap50: Rhaponticum carthamoides extract standardized to 50% content in ecdysteroids. PPM: parts per million.

FIG. 7 shows the increased protein synthesis measured by incorporation of radiolabeled leucine (³H-Leu) with different concentrations of Ajuga turkestanica vs. a control group. DPM: decays per minute. TP: total protein. ATK: Ajuga turkestanica extract standardized to 2% content in ecdysteroids. PPM: parts per million.

FIG. 8 shows the increased protein synthesis measured by incorporation of radiolabeled leucine (³H-Leu) with different concentrations of Cyanotis arachnoidea vs. a control group. DPM: decays per minute. TP: total protein. CyAr: Cyanotis arachnoidea extract standardized to 50% content in ecdysteroids. PPM: parts per million.

FIG. 9 shows the increase in protein synthesis (over a control group) induced by the addition of ascending doses of Rhaponticum carthamoides extract, using 20HE as a reference. The integrated line shows a directly porportional dose response curve up to 20 ppm. RCE: Rhaponticum carthamoides extract. PPM: parts per million.

FIG. 10 shows a comparison of protein synthesis induced by Rhaponticum carthamoides extract, hesperidin or both in combinaiton, measured by the increase in decays per minute per gram of protein (a surrogate marker of protein synthesis in muscle cells) induced by Rhaponticum carthamoides and hesperidin alone or in combination. C−: Negative control, 20HE: 20-Hodroxy-ecdysone, R: Rhaponticum carthamoides extract, H: Hesperidin. Doses are in parts per million (ppm). Y-axis: decays per mimnte per mg of protein. Error bars: Standard deviation.

FIG. 11 is the effect of methandrostenolone on MCF-7 cells. IC₅₀: Inhibitory concentration at 50%. C+: Positive control.

FIG. 12 is the effect of the synthetic androgen methyltrienolone (R1881) on MCF-7 cells. IC₅₀: Inhibitory concentration at 50%. C−: Negative control. C+: Positive control.

FIG. 13 shows the lack of androgenic effects on MCF-7 cells by the addition of ascending doses of Rhaponticum carthamoides and Ajuga turkestanica extracts. C−: Negative control. C+E₂: Positive control with 17β-estradiol. Cₐ: antiandrogenic control with R1881. E₂: 17β-estradiol. ATE: Ajuga turkestanica extract. RCE: Rhaponticum carthamoides extract.

In the above figures, entries marked with “*” show a p-value of <0.05. Entries marked with “**” show a p-value of <0.01. Entries marked with “***” show a p-value of <0.001.

### DETAILED DESCRIPTION

The first observed and classical pharmacological activity of ecdysteroids was their capability of stimulating protein synthesis. This stimulatory effect was reported in the mouse liver. Ecdysteroids isolated from plants were administered orally or intraperitoneally and amino acid incorporation was determined in comparison with an anabolic-androgen (positive control). A protein synthesis increase after 20-hydroxyecdysone administration has been confirmed in both the mouse liver and in the microsomal fraction of the mouse liver.

A number of investigations in the 1970's have involved Ajuga turkestanica, a perennial herb known as a rich source of bioactive substances and used by local people to treat heart diseases, muscle and stomach aches. With regards to ecdysteroids several bioactive compounds have been isolated including turkesterone, 20-hydroxyecdysone (20-HE), cyasterone, cyasterone 22-acetate, ajugalactone, ajugasterone B, α-ecdysone and ecdysone 2, 3-monoacetonide. A characteristic feature of Ajuga turkestanica is the presence of the C11-hydroxylated turkesterone, which has not been observed in other species of the same genus.

Experiments were carried out on sexually mature male rats weighing 200-250 g, sexually immature intact male rate weighing 60-70 g and sexually immature castrated animals (castrated at 40-50 g, administration of the preparation beginning on the day of castration).

Turkesterone and turkesterone tetraacetate as aqueous solutions were administered intragastrically by a special probe at doses of 0.5 mg per 100 g body weight of the animals. The administrations were continued on a daily basis for a period of 10 days. Twenty-four hours after the last administration the animals were sacrificed by decapitation. Anabolic activity of the test substances was assessed and changes in body weight and the weight of the livers, kidneys, hearts and the anterior tibial muscle were also measured along with their total protein content.

Androgenic activity was assessed by the weight of seminal vesicles and ventral prostate. The total protein content of blood serum was determined by refractometry, protein fractions by electrophoresis.

The administration of test substances into sexually mature male rats provoked acceleration in body weight gain. Thus, while control rats gained 5.8 mg per 1 g initial weight per day, the rats receiving turkesterone gained 9.7 mg per 1 g weight per day (2 times greater). Along with this finding, a tendency was noted for weight increases in the liver, heart, kidneys and the Musculus tibialis anterior. There was a statistically significant increase, however, only in heart weight and weight of the anterior tibial muscle in rats receiving turkesterone tetraacetate. In all noted organs there was also an increase in protein content. The protein percentage content was not subject to significant fluctuations. The weight of the ventral prostate and seminal vesicles did not change significantly in this case.

Administration of turkesterone to sexually immature intact rats resulted in weight gain of 38 mg per 1 g initial weight per day; under the influence of turkesterone tetraacetate—37.2 mg/g. Control animals gained 14.7 mg/g during this time. The ratio of test to control in sexually immature rats was 2.6 in the first case and 2.5 in the second. There was a fairly pronounced increase in the weight of organs and a statistically significant increase in total protein content. There was a pronounced effect of turkesterone and turkesterone tetraacetate on the sexual apparatus of rats in this group. Thus, along with an increase in the ventral prostate weight, an increase in seminal vesicle weight was also noted.

In castrated sexually immature rats the manifestations of anabolic activity of the test substances were accompanied by the same patterns noted in intact sexually immature rats, however the weight gain in these animals was less than in the sexually immature intact rats. The ratio of test to control in rats receiving turkesterone was 1.8 and in rats receiving turkesterone tetraacetate—1.9.

The weight of the ventral prostate and seminal vesicles did not change. Total protein content in blood increased, especially in young animals. Similar patterns were observed with respect to albumin as well. Content of α1 and α2—globulins did not change significantly under the influence of turkesterone, but decreased notably after administration of turkesterone tetraacetate. Particularly characteristic was the increase of β- and especially γ-globulins. The increase in the latter was statistically significant in both adult as well as sexually immature animals.

The data presented demonstrate that administration of turkesterone and turkesterone tetraacetate to animals produces an anabolic effect, which in a series of cases was more pronounced for turkesterone tetraacetate.

In a follow up experiment the potential for Turkesterone to increase protein synthesis was assessed directly in an animal model. Briefly, the experiments were carried out on mongrel male mice weighing 18-22 g. Turkesterone (extracted from Ajuga turkestanica) at a single dose of 0.5 mg/100 g and Nerobol (a synthetic anabolic drug) at a dose of 1.0 mg/100 g were administered intragastrically at 1, 2, 4, and 8 hr prior to decapitation, depending on the goal of the study. In experiments with actinomycin D, the antibiotic was administered 30 minutes prior to administration of the test substances at a dose of 2 mg/kg. Polyribosomes from the livers of mice were extracted and functional activity of polyribosomes was studied using a non-cellular protein synthesis system. The polysomal material was analyzed on a saccharose density gradient of 10-50% with a 2.3 M saccharose sublayer. 5-7 mg of polysomes in a volume of 0.5 ml were layered onto the gradient and centrifuged at 26,000 rpm in a SW-30 rotor and a preparatory ultracentrifuge VAC-601 for 120 min. After this, the gradients were distributed by 20 drops into test tubes, adding 2.0 ml water and subjected to spectrophotometrical analysis at 260 nm.

The in vivo experiments used a mixture of ¹⁴C-leucine and valine. Radio-labeled amino acids (740 Bq) were administered to mice 10 minutes prior to decapitation, after which the livers were immediately placed into liquid nitrogen. The addition of ¹⁴C-amino acids and total complete and incomplete proteins in the liver homogenate, as well as the activity of the acid-soluble fraction were determined by precipitation of the radioactive sample, transfer to Millipore filter, and elution with 50 ml of 5% TCA, drying under incandescent lamps, and measuring by a gas-flow meter.

A single-dose administration of turkesterone and Nerobol increased the radioactivity of liver proteins by 1.9-2 times. The activity of the acid-soluble fraction, however, did not change; consequently, the concentration of intracellular ¹⁴C-amino acids in all cases remained the same, and the stimulation by the inclusion of radioactive tags into the acid-insoluble fraction indicated the activation of protein synthesis in mouse liver.

The stimulation of protein synthesis in a non-cellular system resulted from changes only in the polyribosomal apparatus of liver cells. Thus, when testing “experimental” polysomes with cellular fluid derived from the livers of control animals, a clear increase in the functional activity of polyribosomes was observed, which was particularly expressed 4 hours after administration of the preparations.

The activity of cellular fluid in the experiments conducted did not change substantially, however it should be noted that in the system of polyribosomes and cellular fluid from the livers of animals receiving Nerobol, protein synthesis increased by 14% (P<0.001). Upon investigation of the polyribosomal profile of normal cells and those exposed to the activity of the preparations, it was shown that stimulation of the functional activity of liver cell polysomes by turkesterone is not accompanied by substantial changes in the distribution of ribosomes in the saccharose density gradient. Administration of Nerobol brings about an increase in the polyribosomal portion and a decrease in content of dimers and monomers. The ratio of translating to nontranslating ribosomes in the control group is 3.98; after turkesterone administration—3.80; and after Nerobol administration it rises to 7.48.

Stimulation of functional activity and changes in the polyribosomal profile, especially after administration of Nerobol suggested these preparations might activate the transcription of and intensify the synthesis of mRNA or that the effect of these preparations is linked to their effect on translational processes.

Both preparations intensified protein synthesis in the liver by the route of accelerating the synthesis of protein molecules. Experiments with the inhibitor of the synthesis of DNA-dependent RNA, actinomycin D, showed that it almost totally removed the effect of Nerobol, while having no noticeable effect on the stimulatory action of turkesterone. In connection with this it can be supposed that the basis of the stimulatory action of turkesterone and Nerobol on protein synthesis in the liver is explained by different mechanisms. The activity of turkesterone, apparently, was not related to an effect on transcription processes, but more probably is a consequence of activation of protein synthesis processes occurring in the cytoplasm, that is, on the translational level.

Thus, Turkesterone and Nerobol (following single-dose administration in mice) stimulated protein synthesis in vivo and in vitro: in animal livers there was an increase in functional activity of polyribosomes and in conjunction with this, an acceleration of the rate of protein synthesis. Intensification of this protein synthesis under the influence of turkesterone was a consequence of the activation of protein synthesis processes on the translational level. The stimulation of protein biosynthesis by Nerobol was apparently linked to RNA synthesis.

Experiments with doses of 5 mg/kg BW of 20HE derived from Rhaponticum carthamoides showed increases in the weight of liver, heart, kidneys and the tibialis anterior muscle in rats fed for 7 days. Moreover, growth promoting effects were also published independently previously in Japanese quails and pigs.

The above described in vivo experiments set the grounds for the claims sought. Ecdysteroids contained in plant extracts (i.e. Ajuga turkestanica, Rhapontiucm carthamoides or Cyanotis arachnoidea) can be effectively extracted for comprising a pharmacologically active formula with bio available active compounds. Administration of a bioactive extract of the plant induces increases in protein synthesis and lean mass in experimental animals. These effects are similar to synthetic anabolic drugs but their routes of actions may differ. Since the biological action is carried out by the main bioactives in the plant extract (ecdysteroids), extracts obtained from other plants rich in these compounds should have a comparable effect.

Different mechanisms of actions may mean different safety profiles. Those side effects mediated by the same mechanisms of actions of the synthetic steroids can be overcome by molecules with alternative metabolic routes (ecdysteroids being one example). Additionally, aiding in protein synthesis is a desirable effect when treating loss of mass and strength of muscles in sarcopenia, as muscles are mostly made of (contractile) proteins. These aspects of the claims are further developed in the following examples.

Extraction from Plants

Edysteroids and/or their derivatives may be obtained in their isolated form or as an extract containing other compounds from all available natural sources. Alternatively they may be produced synthetically though chemical synthesis. A number of ecdysteroids are commercially available. For instance, alpha-ecdysone and beta-ecdysone are available from CHROMADEX under the references ASB-00005010-001 and ASB-000050015-001 respectively. Other ecdysteroids are currently available from SIGMA ALDRICH and EXTRASYNTHESE.

Natural sources of ecdysteroids and ecdysteroid related compounds include vascular plants, algae, fungi, non-crustacean marine organisms, insects, and other organisms such as nematodes, cestodes and trematodes.

Ecdysteroids are preferentially obtained from plant sources through an extraction process. The concentration of ecdysteroids varies from one species to another and even between varieties of the same species. Ecdysteroid content is generally expressed as percentages by weight of dry matter.

Exemplary processes for the extraction of ecdysteroid, preferably ecdysterone, from plants are described below.

A. Extraction from Plants—General Procedure

In a general procedure for obtaining an ecdysteroid extract, a dried powder of a suitable plant containing ecdysteroids, such as spinach (Spinacia oleracea), Rhaponticum carthamoides, Ajuga turkestanica, Ajuga reptans, or Cyanotis arachnoidea, was extracted in a solvent or solvent mixture, which may be water, a water-miscible organic solvent, or a mixture thereof. The water-miscible organic solvent may be methanol, ethanol, n-propanol, isopropanol, acetone, glycerol, DMSO, ethylene glycol, tetrahydrofuran, dimethylformamide, acetonitrile, or acetic acid. Extraction is carried out for a period of from 2 hours to 48 hours, from 4 hours to 40 hours, from 8 hours to 30 hours, or from 12 hours to 24 hours to produce an extract solution.

The extract solution is optionally concentrated with removal of all or part of the solvent or solvent mixture. After concentration, the resulting extract is resuspended in water.

The extract mixture or the extract solution is then partitioned with a water-immiscible organic solvent, which may be a water-immiscible, nonpolar organic solvent or a water-immiscible ester or alcohol. Suitable water-immiscible, nonpolar organic solvents include, but are not limited to, hexane, heptane, and methylene chloride. Suitable water-immiscible, nonpolar esters or alcohols include, but are not limited to, ethyl acetate and n-butanol.

The organic phase is concentrated by evaporation. The resulting extract is chromatographed on silica gel with a chloroform/methanol mixture. After recrystallization in an ethanol/ethyl acetate mixture, ecdysteroids are obtained in the form of colourless needles.

In some embodiments, the extract mixture or the extract solution is fractionated by sequential extraction after resuspension in water. The aqueous extract mixture or extract solution is partitioned by extraction with a water-immiscible, nonpolar organic solvent, such as hexane, heptane, or methylene chloride. The aqueous fraction is concentrated, and extracted a second time with a water-immiscible ester or alcohol, such as ethyl acetate or n-butanol. The ester or alcohol extract is then concentrated by evaporation to obtain the extract. The extract may be dried and used directly. Alternatively, the extract may be further purified.

The extract may be further purified by chromatography on silica gel with a chloroform/methanol mixture. After elution, the eluent may be recrystallized from an ethanol/ethyl acetate mixture. After recrystallization, ecdysterone is obtained in the form of colourless needles.

B. Extraction from Spinach with Aqueous Alcohol

Locally grown dried spinach powder (S. oleracea) was extracted in 95% ethanol for 24 h. After the removal of ethanol, the extract was resuspended in water and partitioned with heptane. The organic phase was removed, and the water phase was partitioned with butanol. The butanolic phase was dried and used for testing.

C. Extraction from A. Turkestanica with Aqueous Alcohol

The dried aerial portion of A. turkestanica was extracted in 70% ethanol. The ethanolic fraction was removed in vacuo, and the remaining aqueous phase was dried and used for testing.

D. Extraction of Ecdysteroids Using Methanol

A fresh plant containing ecdysteroids is macerated in 5 times its weight of methanol and the mixture is homogenized and filtered. This operation is repeated once more. The extracts are concentrated, and water is added to form a 30% methanol/water solution.

This solution is extracted with hexane. The 30% methanol fraction is concentrated again and extracted with ethyl acetate. The aqueous fraction is extracted with n-butanol. After drying, the resulting n-butanol extract may be used for testing.

Alternatively, the butanol extract may be subjected to further purification.

The butanol extract may be concentrated by evaporation and thereafter treated by chromatography on silica gel with a chloroform/methanol mixture. After recrystallization in an ethanol/ethyl acetate mixture, ecdysterone is thereby obtained in the form of colourless needles.

E. Acetylation of Ecdysteroids

It is also possible to obtain monoacetylated derivatives of ecdysteroids by chemical methods well known to a person skilled in the art. For example, the ecdysteroid is brought into connect with a 1:5 by weight mixture of acetic acid and pyridine at room temperature. Reaction is generally allowed to take place for a period of 30 min to 1 h. The reaction is then stopped by adding methanol. A mixture of different monoacetates of the ecdysteroid is thereby obtained, which products may then be separated conventionally by chromatography. By this process, it is, for example, possible to prepare ecdysterone monoacetates at positions 2, 3, 22 and 25, respectively.

F. Industrial Extraction

The extraction of phytoecdysteroids at an industrial level will generally involve the use of polar solvents such as, but not limited to, the following: methanol, ethanol, propanol, butanol, ethyl acetate, acetone, acetonitrile or tetrahydrofuran. Ethanol/water mixtures containing 20-70% ethanol may be used as the extraction solvents.

### EXAMPLE

A fresh or dried plant containing ecdysteroids is macerated in from 1 to 20 times its weight, from 2 to 10 times its weight, from 2 to 8 times its weight, or 4 times its weight, with an extracting solvent. The mixture is macerated for a period of 0.1 to 72 hours, from 0.5 to 24 hours, from 1 to 12 hours, from 0.75 to 6 hours, from 1 to 3 hours, or 1.5 hrs, with or without mixing, at a temperature ranging from 20 to 70 degrees Celsius, from 25 to 60 degrees Celcius, from 30 to 50 degrees Celcius, or 40 degrees Celcius. The raw material is then separated from the extraction solvent to produce an extract solution by decantation, filtration, centrifugation, or another mechanical means.

In certain embodiments, at least part of the extraction solvent may be removed from the extract solution by evaporating the solvent to concentrate the mixture. When desired, the ecdysteroids present in the extraction solvent may be purified by means of micro/ultrafiltration systems, crystallization, adsorption resins or other means. The extract may be dried by vacuum evaporation, spray drying, freeze drying or any other suitable dehydration or solvent removal method.

### Use of Antioxidants to Preserve Shelf Life

In order to preserve the shelf life of the ecdysteroid extract the formulation may include food grade antioxidants such as, but not limited to: E300 Ascorbic acid, E301 Sodium ascorbate, E302 Calcium ascorbate, E304 Fatty acid esters of ascorbic acid, E306 Tocopherols, E307 Alpha-tocopherol, E308 Gamma-tocopherol, E309 Delta-tocopherol, E310 Propyl gallate, E311 Octyl gallate, E312 Dodecyl gallate, E315 Erythorbic acid, E316 Sodium erythorbate, E319 Tertiary-butyl hydroquinone (TBHQ), E320 Butylated hydroxyanisole (BHA), E321 Butylated hydroxytoluene (BHT), E392 Extracts of rosemary, E586 4-Hexylresorcinol. The amount of such antioxidants used in the formulation will be dictated by the regulatory authorities.

### Ecdysteroid Content

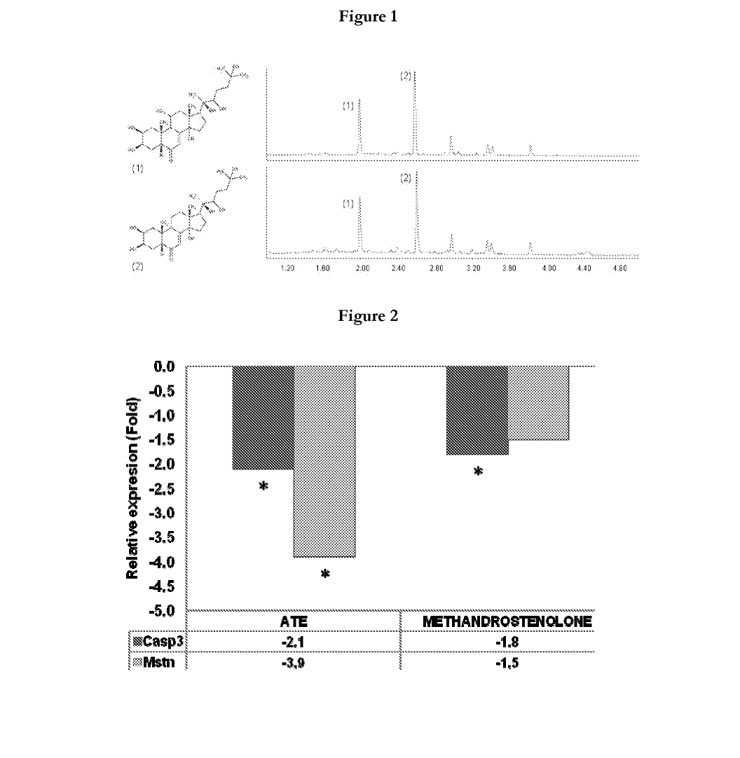

The main ecdysteroids in extracts of Ajuga turkestanica include, but are not limited to: 20HE, turkesterone, and cyasterone. FIG. 1 shows UPLC chromatograms of (A) an extract of the Ajuga turkestanica whole plant, and (B) the corresponding freeze-dried powder extract, measured at 245 nm. FIG. 1 also shows the chemical structures of the two most abundant ecdysteroids, specifically turkesterone and 20-Hydroxyecdysone. The extract of the Ajuga turkestanica whole plant was obtained by extraction in 70% ethanol. The ethanolic fraction was removed in vacuo and the remaining aqueos phase was dried.

The main ecdysteroids in extracts of Rhaponticum carthamoides include, but are not limited to: 20HE, inokosterone, 24-epi-makisterone, ajugasterone C, polypodine B, and makisterone A. The main ecdysteroids in extracts of Ajuga turkestanica include, but are not limited to: 20HE, turkesterone, and cyasterone. The main ecdysteroids in extracts of spinach include, but are not limited to: 20HE and polypodine B.

The total ecdysteroid content in an extract of spinach, Ajuga turkestanica, Rhaponticum carthamoides, or Cyanotis arachnoidea may be between 0.1 and 90%, or between 0.1 and 5%. The amount of 20HE in the extracts can range from 0.1 to 90%, or between 0.1 and 5%.

Cyanotis longifolia contains ecdysteroids, which are highly concentrated in the roots and flowers, while leaves contain lesser amounts and stems intermediate amounts. The main ecdysteroids in extracts of Cyanotis longifolia include, but are not limited to: 20HE, 20-hydroxyecdysone 3-acetate, ajugasterone C, polypodine B, 2-deoxy-20,26-dihydroxyecdysone, isovitexirone, and poststerone.

### Example 1

Ajuga turkestanica Extract Inhibits Genes that are Over Expressed in Sarcopenia.

The effect of Ajuga turkestanica extract (ATE) standardized to Turkesterone content was studied in muscle cells. A mouse skeletal muscle cell line, C2C12 (American Type Culture Collection, U.K), was cultured in Dulbecco's Modified Eagle's Medium (DMEM) with high glucose (Thermo Fisher Scientific, Spain) supplemented with 10% fetal bovine serum (Lonza Group, Switzerland), 2 mM glutamine, 100 units/ml penicillin and 100 μg/ml streptomycin. Cells between passages 3 and 10 were seeded at a density of 10,000 cells per cm². Cells were grown for 48 h until they reached 80-90% confluence. To induce myogenic differentiation, the medium was replaced with differentiation medium, DMEM supplemented with 2% horse serum (PAA Laboratories, Austria). After 10 days the myoblasts had fused into multinucleated myotubes. Cells were maintained at 37° C. in a humidified 5% CO2 incubator and medium was changed every other day.

For RNA extraction, C2C12 cells were plated and differentiated to myotubes into 12-wells plates. After differentiation, cells were incubated with 20 ppm ATE (approx. 1 μM total ecdysteroids) or 1 μM methandrostenolone for 6 h. RNA was then extracted using an All Prep RNA/Protein Kit (Qiagen, Spain). Total RNA was quantified using a fluorometric method with Quant-iT kit (Invitrogen, Spain). RNA was stored at −80° C. until further use. cDNA was reverse-transcribed from the RNA extract using RT² First Stand cDNA kit and we used a RT² Profiler PCR Array to analyze a panel of 84 genes involved in skeletal muscle development and disease (Qiagen, Spain). Quantitative real-time RT-PCR was carried out using a SYBR-Green/ROX detection in a MX3005P Q-PCR System. Samples were heated at 95° C. for 10 min, followed by a second stage composed of 15 sec at 95° C., 1 min at 60° C. which was repeated 40 times and third stage for dissociation curve composed of 1 min at 95° C., 30 sec at 55° C. and 30 sec at 95° C. The PCR-array data was analyzed by calculating relative gene expression and statistical significance through the use of known methods.

The addition of ATE resulted in a statistically significant down-regulation of caspase-3 (2-fold) and Myostatin (4-fold), compared with methandrostenolone (<2-fold both genes). Results are shown in FIG. 2.

During muscle wasting caspase-3 activation and the ubiquitin proteasome system (UPS) act synergistically to increase the degradation of muscle proteins. Activation of the former is required to convert actomyosin and myofibrils into substrates of the UPS. Caspase-3 cleaves specific 19 S proteasome subunits in C2C12 muscle cells with a cell-specific activity. Caspase-3 cleaves different subunits in myoblasts and myotubes hence intervening in cell differentiation or muscle wasting. Recently, it has shown that adding a caspase-3 inhibitory peptide to myotube cultures resulted in inhibition of tumor necrosis factor-like weak inducer of apoptosis (TWEAK) induced loss of myosin heavy chain and myotube diameter. Myostatin is mostly expressed in skeletal muscle and normally functions as a negative regulator of muscle growth. Upon the binding to activin type IIB receptor, this extracellular cytokine initiates several different signaling cascades resulting in the down-regulation of the important myogenesis genes. Muscle size is regulated via a complex interplay of myostatin signaling with the insulin-like growth factor 1/phosphatidylinositol 3-kinase/Akt pathway responsible for increase in protein synthesis in muscle. Thus, myostatin blockage or its natural absence leads to a significant increase in muscle mass.

### Example 2

Ajuga turkestanica Extract Modulates Muscle Cell Differentiation Via the Expression of Myomesin1

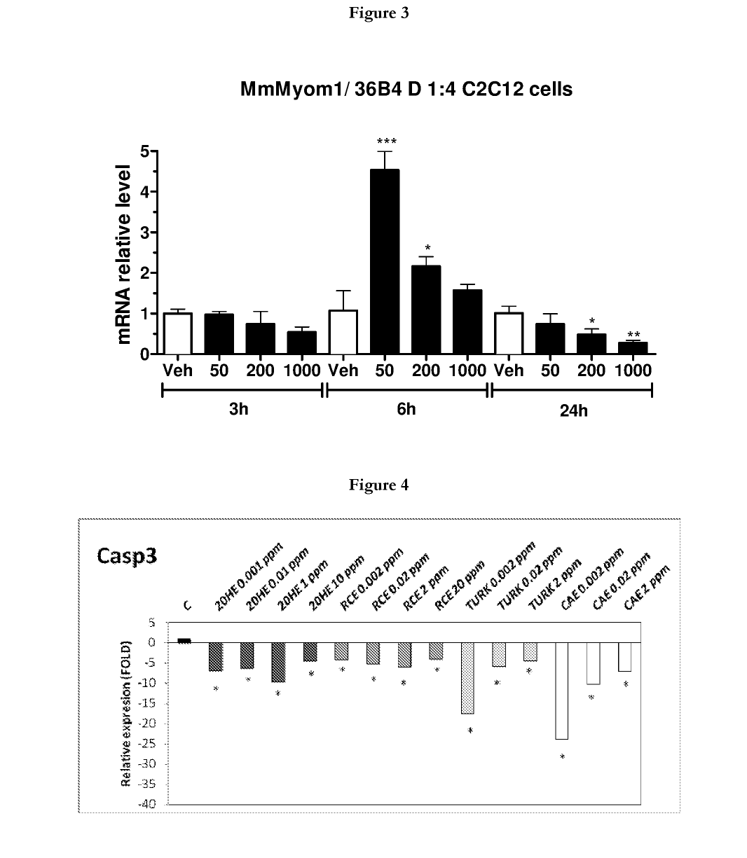

In this experiment, C2C12 cells were cultured and differentiated as previously described. An average of 10,000 cells/cm² was seeded and incubated for 48 h (viability 94%). Then, growth media was changed for differentiation media which was replaced every 48 h until day 8. Quantitative RT-PCR was performed after adding increasing concentrations of ATE to the media (50, 200 and 1000 μg/ml) at day 8. Myomesin 1 (myom1) showed statistically significant unregulated levels of expression compared with the negative control (vehicle) at 6 and 24 h of treatment (FIG. 3).

Myomesin 1 belongs to the proteins that form the sarcomeric skeleton of muscle cells along with titin, C-protein, α-actinin, and M-protein and is present in both slow and fast fibers in the muscle. Sarcomeres (the structural units that produce contractions in muscles) have at their centre a side-by-side array of bipolar thick filaments formed mainly of myosin (a motor protein). This thick filament array is interlinked at the middle by bridging proteins forming the M-band. Interposed with thick filaments from both sides are thin filament arrays made mainly of actin, which are cross-linked by the Z-band proteins. Myomesin 1 is mostly located in the centre of the M-band. Myomesin 1 functions as a molecular bridge that connects major filament systems in the central M-band of muscle sarcomeres, thus been responsible for passive stress sensing. Demonstration of upregulated expression of this gene induced by ecdysteroids suggests their role in muscle health and/or maintenance.

### Example 3

Ecdysteroids Contained in Ajuga turkestanica Increase Protein Synthesis in Muscle Cells.

A mouse skeletal muscle cell line, C2C12 (ATCC CRL-1772), was maintained according to the usual method. Between passages 3 and 10, cells were seeded at a density of 10⁵ cells/cm² onto 24-well tissue culture plates. The cells were grown in low-glucose Dulbecco's Modified Eagle's Medium (DMEM) supplemented with 10% fetal bovine serum (FBS), 10 mM HEPES, 6 mM glutamine, 1 mM pyruvate, 100 units/mL penicillin, and 100 μg/mL streptomycin (Gibco, Grand Island, N.Y.). Cells were grown for 48 h in 5% CO₂ at 37° C. After cells reached 80% confluency, the medium was replaced with differentiation medium (DMEM with 2% horse serum). After 5 days, the myoblasts had fused into multinucleated myotubes. Primary human skeletal muscle cells (a gift from Dr. William Cefalu of the Pennington Biomedical Research Center, Baton Rouge, La.) were seeded at a density of 105 cells/cm2 onto 24-well tissue culture plates. The cells were grown in DMEM supplemented with 10% FBS and a SingleQuot Kit (Lonza, Portsmouth, N.H.) containing epidermal growth factor, insulin, bovine serum albumin (BSA), fetuin, dexamethasone, and gentamicin/amphotericin-B. Cells were grown for 96 h in 5% CO₂ at 37° C. until they reached 80% confluency, and the medium was replaced with differentiation medium (DMEM with 2% horse serum). After 18 days, myoblasts fused into multinucleated myotubes. Cells were treated with increasing concentrations of 20-Hydroxyecdysone (20HE), Turkesterone, ponesterone, polypodine B, methandrostenolone, or vehicle. Additionally, extracts of spinach or Ajuga turkestanica were also added. Radio-labeled leucine was used to assess any changes in protein synthesis. Decays per minute (DPM) were measured in a liquid scintillation counter (LS 6500, Beckman Coulter). Total protein was quantified using the bicinchoninic acid (BCA) method following the manufacturer's instructions (Pierce, Rockford, Ill.). The data were expressed as DPM per milligram of total protein.

All of the tested ecdysteroids increased protein synthesis in C2C12 myotubes in a dose-dependent manner. 20HE and turkesterone elicited the strongest response, increasing protein synthesis to 110% of control at 40 nM. This effect peaked at 0.1 μM, with protein synthesis at 120% of control and was still observed at concentrations up to 10 μM. Methandrostenolone, an anabolic steroid, had no significant effect on protein synthesis at concentrations up to 10 μM. In human skeletal myotubes, 20HE produced a similar dose dependent increase in protein synthesis after 24 h of treatment. Treatment with 100 nM 20HE increased protein synthesis by 120% of control. This increase was observed at up to 1 μM.

Additionally incubating cells with a phosphoinositide kinase-3 inhibitor significantly reduced the effect on protein synthesis, which indicates the action of the ecdysteroids could use this molecular pathway as also demonstrated in example 1.

### Example 4

Other Ecdysteroid Rich Extracts from Plants (Rhaponticum carthamoides and Cyanotis arachnoidea) Down Regulate the Same Molecular Pathways Involved in Sarcopenia

Experiments similar to those in example 1 have been carried out with two other plant-derived, ecdysteroid-rich extracts. The same mouse skeletal muscle cell line, C2C12 (American Type Culture Collection, U.K), was again cultured in Dulbecco's Modified Eagle's Medium (DMEM) with high glucose (Thermo Fisher Scientific, Spain) supplemented with 10% fetal bovine serum (Lonza Group, Switzerland), 2 mM glutamine, 100 units/ml penicillin and 100 μg/ml streptomycin. Cells between passages 3 and 10 were seeded at a density of 10,000 cells per cm². Cells were grown for 48 h until they reached 80-90% confluence. To induce myogenic differentiation, the medium was replaced with differentiation medium, DMEM supplemented with 2% horse serum (PAA Laboratories, Austria). After 10 days the myoblasts had fused into multinucleated myotubes. Cells were maintained at 37° C. in a humidified 5% CO2 incubator and medium was changed every other day.

Cell viability after adding the active compounds and extracts was determined using the Presto Blue cell viability kit (Invitrogen, Spain) following the manufacturer's instructions and following the same steps as in example 1.

For RNA and protein extraction C2C12 myotubes were incubated with 0.002, 0.02 and 2 ppm of Rhaponticum carthamoides extract (RCE) and Cyanotis arachnoidea extract (CAE) or 0.001, 0.01 and 1 ppm of 20-hydroxyecdysone or 0.002, 0.02 and 2 ppm of Turkesterone for 24 hours. These two latter served as positive controls. RNA and protein were extracted using All Prep RNA/Protein Kit (Qiagen, Spain). RNA was stored at −80° C. and protein at −20° C. until further use. Total RNA was quantified using a fluorometric method with Quant-iT kit (Invitrogen, Spain). The cDNA was reverse-transcribed from the RNA extract using RT²First Stand cDNA kit (Qiagen, Spain). Quantitative real-time RT-PCR for Caspase-3 and Myostatin were carried out using a MX3005P Q-PCR System (Agilent Technologies) and SYBR-Green/ROX detection. The housekeeping gene used was Actb and Mouse XpressRef Universal total RNA (Qiagen, Spain) as a positive control. Samples were heated at 95° C. for 10 min, followed by a second stage composed of 15 sec at 95° C., 1 min at 60° C. which was repeated 40 times and thirst stage for dissociation curve composed of 1 min at 95° C., 30 sec at 55° C. and 30 sec at 95° C. To control the quality of PCR reactions, no-cDNA template control and no-reverse transcription control samples were included. The fold change in gene expression was calculated with ΔΔCt Method.

Results of the gene modulation experiments are depicted in FIGS. 4 and 5. Both RAE and CAE induced statistically significant down regulation of Caspase 3 (which, as stated above is upregulated in sarcopenia). Equivalent doses of CAE (in ppm) seemed to have a greater effect on gene modulation than that of RAE. While there was little variation between doses of RAE, ascending doses of CAE had a descending effect on gene inhibition. Both positive controls (20HE and turkesterone) also had a significant effect in down regulation of Caspase 3 (FIG. 4).

In terms of Myostatin modulation, only the highest dose of RCE (20 ppm) showed a statistically significant down regulation. Interestingly, CAE showed a dose response curve where all concentrations effectively shut down Myostatin (FIG. 5). Again, these results were concordant to the effect of both standards (20HE and turkesterone, both bioactives present in the extracts in various rations) (FIG. 5). Muscle size is directly inhibited by Myostatin, and so down regulation of this gene is expected to have a positive effect in muscle growth.

In conclusion these experiments showed that bioactive ecdysteroids retained after extracts are obtained from the plants elicit a biological response similar to that of Ajuga turkestanica extract.

### Example 5

Extracts Derived from Ajuga turkestanica (ATE), Rhaponticum carthamoides (RCE) and Cyanotis Arachnoidea (CEA) Stimulate Protein Synthesis in Muscle Cells.

In further experiments, myotubes were again obtained by culturing C2C12 cells and allowing their terminal differentiation. This cell line was selected on their ease of use, robustness, and similarity to skeletal muscle tissue. These cell can be grown indefinitely as undifferentiated myoblast, and when needed can be differentiated rapidly into multinucleated myotubes which behave in many ways like skeletal muscle fibers, contracting when stimulated and expressing characteristic muscle protein like myogenin, myosin heavy chains and androgen receptor. The goal of the study was to demonstrate a biological, measurable effect derived from the modulation of molecular pathways involved in maintaining/improving muscle tissue quality that could relate directly to ameliorating sarcopenia.

C2C12 myoblasts were obtained from the American Type Culture Collection (ATCC, CRL-1772) and grown routinely on Culture Flasks T-75 (Corning) in Growth Medium (GM): Dulbecco's modified Eagle medium (DMEM) (HyClone; Thermo Fisher Scientific) supplemented with 10% fetal bovine serum (FBS) (Biowest), 10 mM Hepes (Lonza), 6 mM L-glutamine, 100 U/mL penicilin and 100 μgr/mL streptomycin (Biochrom) in a humidified atmosphere of 95% air and 5% CO at 37° C.

Cells between passages 3 and 10 were seeded in GM at a density of 10⁴ cells/cm² onto 24 well tissue culture plates (Corning). After cells reached 80-90% confluence (72 hours post-seeding) the media was replaced with differentiation media (DM): DMEM containing 2% horse serum (PAA; GE HealthCare), 10 mM Hepes (Lonza), 6 mM glutamine, 100 U/mL penicilin and 100 μgr/mL streptomycin (Biochrom). Media was replaced every 2 days, and after 10 days the myoblast has fused into multinucleated myotubes showing a fusion index of 70-80%.

Before cell treatment, a 2000× Botanicals Extracts Stocks solutions were prepared in Biological grade DMSO (Sigma) or Ultrapure H2O (milliQ; Millipore), sonicated for 30 min and Filtered using a 0.22 μm pore size syringe filter membrane (Millipore).

For the botanical extracts dose response, differentiated C2C12 myotubes were washed 3 times with PBS (1.5 mL of medium/well) and treated with increasing concentrations of Rhaponticum carthamoides extract (RCE; 0.005, 0.05, 0.2, 2, 20, 100 and 200 ppm), Ajuga turkestanica extract (ATE; 20, 100 and 200 ppm) and Cyanotis arachnoidea extract (CAE; 2, 20 and 200 ppm), or the vehicle, 0.005% DMSO, four wells per treatment. Positive controls of anabolic activity were 20-Hydroxyecdysone (20HE; 1 and 10 μM equivalent to 0.5 and 5 ppm) and 5α-Dihydrotestosterone (DHT) at the same doses. All Botanical extracts were manufactured by PoliNat (details may be found in section entitled “Extraction from plants”), DHT (99% purity verified by TLC) was supplied by Sigma-Aldrich and 20-HE (95% purity verified by 1H-NMR) was purchased to Enzo Life Sciences. Compounds were added to serum-free DM containing 5 μCi/ml L-[1,3,5-3H]-Leucine. Cells were incubated for 16 h before protein measurement.

Protein synthesis was determined by measuring the incorporation of the tritiated L-Leucine (³H-Leu) into C2C12 cellular proteins. Briefly, following treatment, cells were washed three times with 2 mL of cold phosphate buffered saline (PBS), followed by the addition of 10% of cold trichloroacetic acid (TCA) to precipitate cellular proteins. After 60 min at 4° C., the TCA was removed, the precipitate was washed with cold PBS and dissolved in 0.5 M NaOH (600 μl). The dissolved precipitate (400 μl) was added to scintillation vials with 5 ml of scintillation fluid (Optiphase, Perkin Elmer) and vortex during 20 seconds to mix efficiently. Radioactivity was measured using a liquid scintillation counter (Tri-Carb, Perkin Elmer) and expressed as decay per minute (DPM). Total protein was quantified using Bradford method-based assay (microassay procedure) following the manufacturer's instructions (Biorad protein assay, BioRad Laboratories). Protein synthesis was calculated from the trichloroacetic acid-insoluble radioactivity incorporated per microgram of total protein. Results were expressed as relative increase in L-[3H]-Leucine compared with 100% of not treated Control. Each experiment was performed in triplicate.

All three plant extracts increased protein synthesis to a greater extent than the control group (p<0.05). Increases varied from 32% (±6.74%) for RCE at 200 ppm dose to 85.7% (±19.55%) for CAE at 2 ppm. Compared with the positive control group (20HE at 10 μM), all 3 extracts showed a statistically significant effect at all doses, as shown in Table 1.

When compared with the 20HE standard (at 10 μM or 5 ppm) all extracts showed a more variable degree of increased protein synthesis, with ATE and CAE outperforming RCE, as shown in Table 2.

TABLE 1
Increase in protein synthesis expressed as percentage
over the control of 3 plant extracts.

| | Dose | % Difference in protein | |
|---|---|---|---|
| Plant extract | (ppm) | synthesis (±SD) | |
| Rhaponticum | 2 | 39.09 | (16.35) |
| carthamoides | 20 | 48.34 | (14.26) |
| | 200 | 32.00 | (6.74) |
| Ajuga | 20 | 68.15 | (15.32) |
| turkestanica | 100 | 74.59 | (14.80) |
| | 200 | 60.35 | (9.64) |
| Cyanotis | 2 | 84.47 | (19.55) |
| arachnoidea | 20 | 75.68 | (14.59) |
| | 200 | 41.12 | (9.62) |

All values, p < 0.05.
PPM: parts per million.
SD: Standard deviation.

TABLE 2
Increase in protein synthesis of 3 extracts compared
with the active group (20HE at 5 ppm).

| | Dose | % Difference in protein | |
|---|---|---|---|
| Plant extract | (ppm) | synthesis (±SD) | |
| Rhaponticum | 2 | 4.49 | (14.21) |
| carthamoides | 20 | 10.96 | (5.92) |
| | 200 | −1.03 | (5.27) |
| Ajuga | 20 | 25.82 | (5.26)* |
| turkestanica | 100 | 30.56 | (3.32)* |
| | 200 | 20.74 | (14.66)^{Π} |
| Cyanotis | 2 | 38.12 | (12.01)* |
| arachnoidea | 20 | 31.96 | (13.89)* |
| | 200 | 5.75 | (6.03) |

^{Π}p < 0.01,
*p < 0.001.
PPM: parts per million.
SD: Standard deviation.

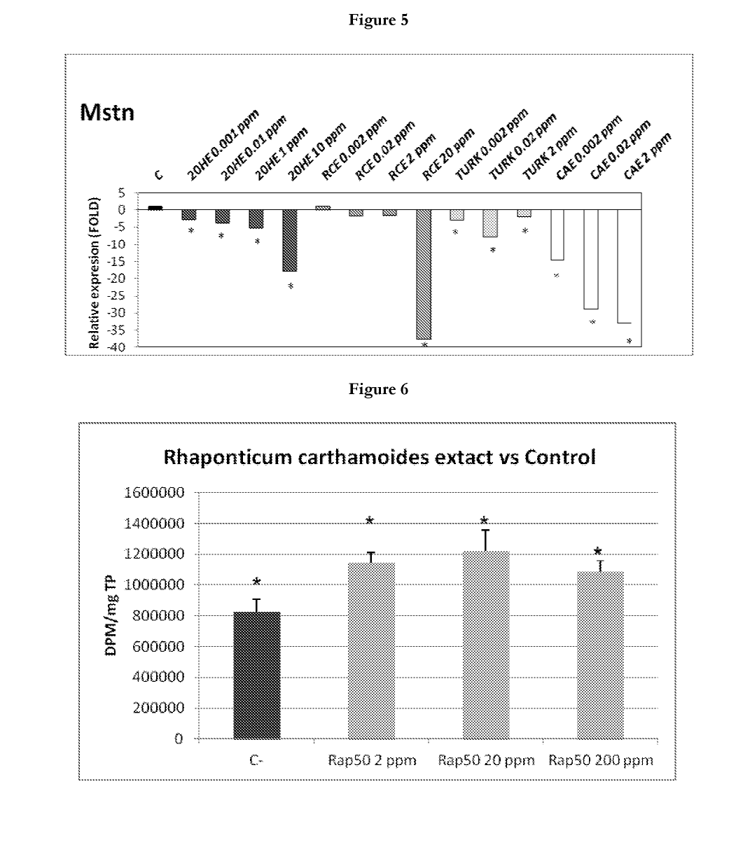

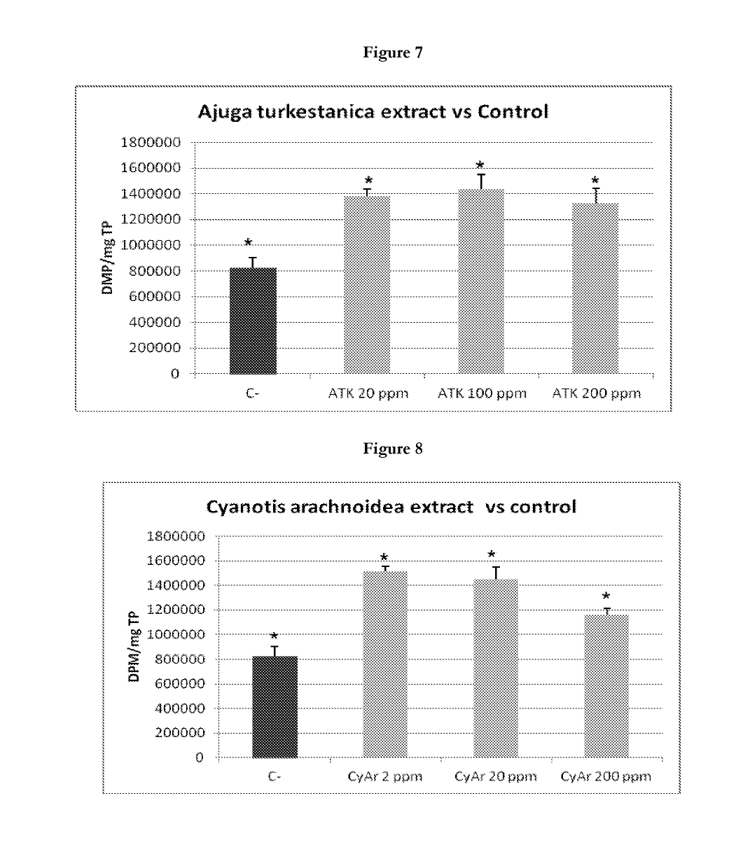

Dose response curves were later obtained by comparing different doses per extract versus ³H-Leu content in proteins. Both RCE and ATE showed a biphasic curve with an ascending arm that plateaus before the 200 ppm dose, hinting at a mechanism that may saturate at high concentrations (FIGS. 6 & 7). Interestingly, CAE showed an indirectly proportional relation between dose and effect (FIG. 8).

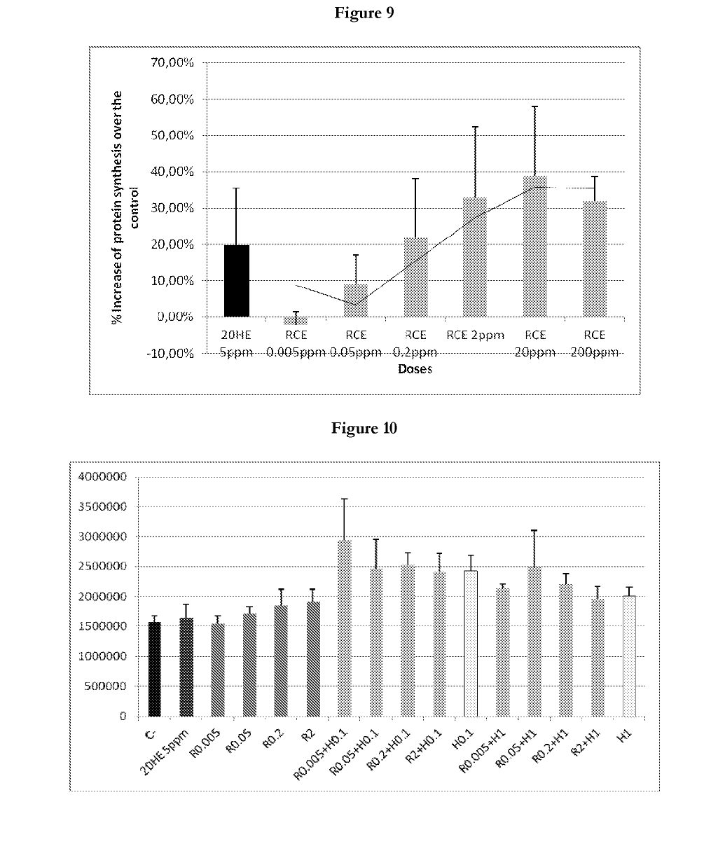

Finally, a dose response curve was constructed for RAE at lower doses previously tested and using 20HE at 10 μM (or 5 ppm) as the positive control. Results are depicted in FIG. 9 and show the percentage increase in protein synthesis displayed a proportional relation at low doses (0.005 ppm to 20 ppm) whereas at higher doses there is probably a saturation of the mechanisms involved (200 ppm).

In conclusion, it was found that ecdysteroid rich extracts obtained from plants (in this case Rhaponticum carthamoides, Ayuga turkestanica and Cyanotis arachnoidea) can elicit an increase in protein synthesis in muscle cells. This effect has biological plausibility for two main reasons; first pure 20HE also produced a statistically significant increase in protein synthesis. This is a compound commonly found in various proportions in these plants and can be concentrated during the manufacturing process as described in section entitled “Extraction from plants”. Secondly, there is a correlation between different doses and the biological effect.

In further experiments hesperidine was added to myotube cultures to assess for possible synergies between plant ecdysteroids and this compound. Briefly, hesperidine is a flavonone glycoside found in citrus fruits with several pharmacological properties (i.e. decreased blood pressure and cholesterol levels in rats). Additionally, hesperidine has shown to stimulate muscle synthesis by up-regulating the expression of myoD (a protein involved in myogenic differentiation) also in animal models. This molecular pathway could be a complementary and/or synergistic novel mechanism to those of Caspase 3 and Myostatin modulation in stimulating protein synthesis in muscle tissue.

Cell cultures were then treated with no extract (negative control), 20HE 5 ppm (positive control), and doses of RCE (0.005, 0.05, 0.2 and 2 ppm) alone or in combination with hesperidin 0.1 and 1 ppm. Protein extraction and quantification were performed as previously described.

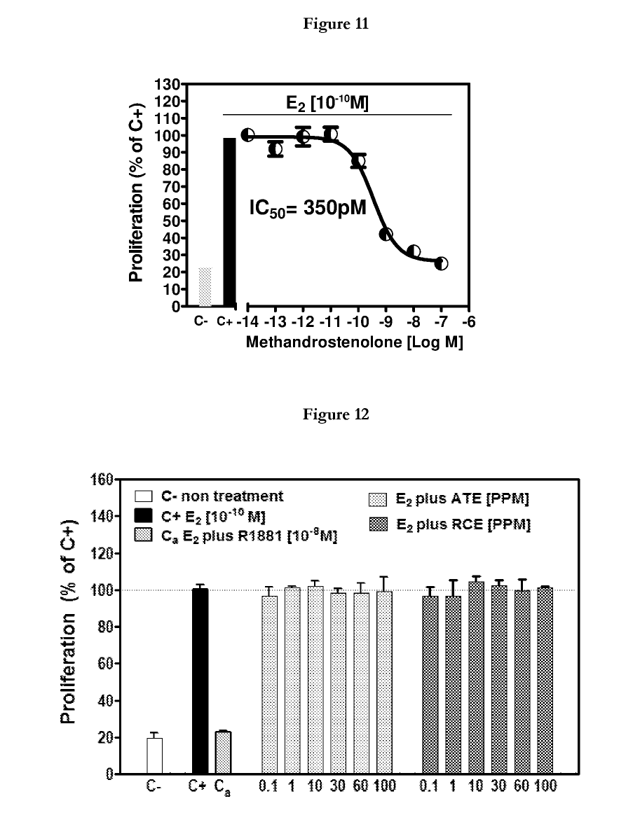

Ascending doses RCE showed an increased protein synthesis compared with control in a directly proportional fashion to the dose but with no statistical significance until RCE was mixed with Hesperidin (FIG. 10). The highest increase was seen with RCE at 0.005 ppm+Hesperidin 0.1 ppm (p<0.0001). The addition of both doses showed a statistically significant increase when compared with both compounds individually (FIG. 10). All mixed doses (RCE+hesperidin) showed statistically significant increase in protein synthesis when compared to control and to a greater extent than RCE alone, as shown in Table 3.

TABLE 3
Comparison of increased protein synthesis induced by
Rhaponticum carthamoides and Hesperidin alone and in combination.

| Comparison | p(2-tail) | %↑ |
|---|---|---|
| R0.005 vs R0.005 + H0.1 | 1.18647E−07 | 90.60% |
| H0.1 vs R0.005 + H0.1 | 0.026474479 | 21.24% |
| R0.05 vs R0.05 + H0.1 | 0.001867434 | 43.03% |
| H0.1 vs R0.05 + H0.1 | 0.869006604 | 1.54% |
| R0.2 vs R0.2 + H0.1 | 0.004668956 | 35.96% |
| H0.1 vs R0.2 + H0.1 | 0.662055188 | 4.08% |

R: Rhaponticum carthamoides extract, H: Hesperidin. Doses are in parts per million.
%↑: Percentage of increased synthesis using negative control to normalize.

### Example 6

Ecdysteroids from Ajuga turkestanica do not Bind Androgen Receptors to Increase Protein Synthesis.

For this example, investigators used an androgen receptor binding assays to compare 20HE with methandrostenolone, at increasing concentrations. Briefly, recombinant rat androgen receptor was combined with [3H]mibolerone in a buffer of 50 mM Tris-HCl, pH 7.5, 0.8 M NaCl, 10% glycerol, 2 mM dithiothreitol, 1 mg/mlL BSA, and 2% ethanol. DPM of the incubation buffer was measured to quantify displacement of the labeled ligand. Each treatment was repeated four times, and the results were averaged. Concentrations of 1 to 100 μM 20HE did not show any significant inhibition while concentrations of 0.01 to 1.00 μM of methandrostenolone showed a dose dependent inhibition up to 94%.

### Example 7

Ajuga turkestanica and Rhaponticum carthamoides Extracts (ATE and RCE) Rich in Ecdysteroids do not Show Androgenic Activity in Cell Cultures.

In this example ATE and RCE were assessed for androgenic activity using the A-screeen assay. MCF7-AR1 is a human cancer-derived cell line which has been genetically engineered to overexpress the androgenic receptor (AR). The A-Screen cell bioassay, developed to measured anti-androgenic activity using MCF7-AR1 cell number as the end point, is used to identify androgenic chemicals among environmental pollutants and it has proved to be very sensitive and reproducible assay for detecting androgenic a. This assay measures androgen-dependent inhibition of proliferation of the androgen receptor (AR)-positive human mammary carcinoma cell line, MCF7-AR1. This cell line has been stably transfected with a full human AR and expresses approximately five times more AR than wild-type cells. MCF7-AR1 cells retain the capacity to proliferate in response to estrogen treatment (E2). Androgens inhibit estrogen-induced proliferation and cells arrest in G0/G1 phase in a dose-dependent manner.

Since androgenic activity is ultimately based on cell number end-point, it was necessary to establish whether any decrease in proliferation could be associated with a cytotoxic effect of the extract rather than AR agonist action. Trypan blue dye exclusion assay was used to examine extract-mediated cytotoxicity (expressed as non-viable cells) and to assess cell viability upon exposure to them in complete medium (FBS-DMEM). ATE and RCE treatments reduced both total cell number and viability of MCF7-AR1 cells in a dose-dependent manner (data not shown). The results show that ATE and RCE both inhibit MCF7-AR1 cell viability at very high doses (≥200 ppm) by inducing both a cytotoxic cell response and reducing the number of viable cells. However, based on the above the concentration of ATE and RCE used in the A-Screen assay was limited to a range of 0.1-100 ppm.

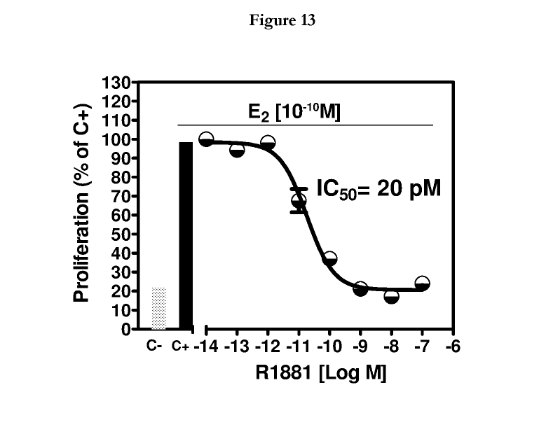

The A-Screen bioassay compares the cell number of similar inocula of MCF-7-AR1 cells growing in media in the absence of any estrogen and androgens (C−, negative control), in the presence of E2 (C+, estrogen control) and in the presence of E2 in combination with different concentrations of the suspected androgen (FIG. 11). Androgenic activity of a test compound results in the inhibition of cell proliferation compared to the E2 control. The synthetic androgen methyltrienolone (R1881) was used as the reference compound (Ca, positive control) and a dose-response curve showed that R1881 inhibited cell proliferation at very low concentrations (FIG. 12, IC50=20 pM). Methandrostenolone also inhibited cell proliferation, but at higher concentrations (FIG. 4b, IC50=350 pM). However, adding ATE or RCE at a range of concentrations (0.1-100 ppm) to the culture media in the presence of E2 did not show a significant proliferative inhibition compared with the control (MCF7-AR1 cells plus E2) at doses up to 100 ppm (FIG. 13). Based on these results, it was established that ATE and RCE do not show androgenic activity within the dose range used.

The following examples relate to combinations of:
i) A combination of 20HE+at least one specific ecdysteroid; and
ii) at least one other active ingredient (coadjuvant). By using multiple ingredients effective in treating sarcopenia or symptoms thereof, synergistic or otherwise enhanced efficacy may be achieved. Some of the selected coadjuvants that may be used show anti-inflammatory activity, including Zingiber officinale, Curcuma longa, Ginkgo biloba, Panax ginseng, IGF-1, and Echinacea. Other coadjuvants activate satellite cell activity (Citrus sinensis). Some coadjuvants are bio-enhancers, increasing the availability of the products in plasma (Piper nigrum, Zingiber officinale, Silybum marianum). Some are associated with cell regeneration, growth, or differentiation (Ginkgo biloba, Vitamin D3, Resveratrol, Dioscorea napponica, Cardo Mariano) or have estrogen-like activity (Opuntia).

### Example 8

Compositions Comprising Curcuma longa Extracts.

The current examples set forth compositions comprising i) a combination of 20HE and at least one ecdysteroid, and ii) an effective amount of an anti-inflammatory extract of Curcuma longa. These combinations may further comprise an additional component in an amount effective to inhibit curcumin glucuronidation. The additional component may be an extract of Piper nigrum, Cardo mariano, or Allium cepa. By inhibiting curcumin glucuronidation, extracts of Piper nigrum, Cardo mariano, or Allium cepa increase curcumin bioavailability. The compositions are set forth in Table 4 below.

TABLE 4
Compositions containing Curcuma longa extracts.

| Example | Steroid | Curcuma longa | Glucuronidation | |
|---|---|---|---|---|
| No. | Component | Component | Inhibitor | Benefits |
| 8A | 20HE + | Curcuma longa extract; | None | Increased |
| | Ecdysteroids | Active ingredients: | | muscle mass; |
| | | curcumin; | | Anti- |
| | | demethoxycurcumin; | | inflammatory |
| | | bisdemethoxycurcumin | | |
| 8B | 20HE + | Curcuma longa extract; | Piper nigrum extract | Increased |
| | Ecdysteroids | Active ingredients: | | muscle mass; |
| | | curcumin; | | Anti- |
| | | demethoxycurcumin; | | inflammatory; |
| | | bisdemethoxycurcumin | | enhanced |
| | | | | curcumin |
| | | | | bioavailability |
| 8C | 20HE + | Curcuma longa extract; | Cardo mariano | Increased |
| | Ecdysteroids | Active ingredients: | extract; Active | muscle mass; |
| | | curcumin; | ingredient: Silibinin | Anti- |
| | | demethoxycurcumin; | | inflammatory; |
| | | bisdemethoxycurcumin | | enhanced |
| | | | | curcumin |
| | | | | bioavailability |
| 8D | 20HE + | Curcuma longa extract; | Allium cepa extract; | Increased |
| | Ecdysteroids | Active ingredients: | Active ingredient: | muscle mass; |
| | | curcumin; | Quercetin | Anti- |
| | | demethoxycurcumin; | | inflammatory; |
| | | bisdemethoxycurcumin | | enhanced |
| | | | | curcumin |
| | | | | bioavailability |

### Example 9

### Compositions Comprising 20HE, Ecdysteroids, and Additional Extracts.

The current examples set forth compositions comprising i) a combination of 20HE and at least one ecdysteroid, and ii) an effective amount of an anti-inflammatory extract of Curcuma longa. The compositions are set forth in Table 5 below. The compositions are useful for treating loss of muscle mass in a patient, and contain an effective amount of a composition comprising 20HE and at least one ecdysteroid, in combination with a bioactive component which may be from 10% to 90%, from 20% to 70%, or from 30% to 50% of:
an extract of Ginkgo biloba;
an extract of Zingiber officinale comprising 6-gingerol, 6-shogaol, 6-paradol, or a mixture thereof, said extract of Zingiber officinale being present in an amount effective to promote recovery of muscle strength following exercise;
an extract of ginseng in an amount effective to delay sarcopenia;
resveratrol in an amount effective to stimulate at least one of i) activation of satellite cells, ii) myogenesis in myscle tissue; and iii) hypertrophy in muscle tissue;
an extract of prickly pear cactus comprising at least one phyto-estrogen;
an extract of Dioscorea napponica comprising dioscin, said extract of Dioscorea napponica being present in an amount effective to promote at least one of osteoblast differentiation and osteoclast inhibition;
a secosteroid hormone; and
an extract of Citrus sinensis in an amount effective to stimulate at least one of i) activation of satellite cells, ii) myoblast differentiation; and iii) osteoblast differentiation, where all percentages are based on the combined weight of 20HE, the ecdysteroid(s), and the bioactive component.

TABLE 5
Compositions comprising 20HE, ecdysteroids, and additional extracts.

| | Steroid | Further Extract | |
|---|---|---|---|
| Example No. | Component | Component | Benefits |
| 9A | 20HE + | Gingko biloba extract | Increased muscle mass; |
| | Ecdysteroids | | Delays sarcopenia |
| 9B | 20HE + | Zingiber officinale extract; | Increased muscle mass; |
| | Ecdysteroids | Active ingredients: | Anti-inflammatory; |
| | | 6-gingerol, 6-shogaol, | Accelerated recovery after |
| | | 6-paradol | exercise |
| 9C | 20HE + | Ginseng extract | Increased muscle mass; |
| | Ecdysteroids | | Delays sarcopenia |
| 9D | 20HE + | Resveratrol | Increased muscle mass; |
| | Ecdysteroids | | Reduced muscle loss; |
| | | | improved regeneration of |
| | | | muscle tissue |
| 9E | 20HE + | Prickly pear cactus extract; | Increased muscle mass; |
| | Ecdysteroids | Active ingredients: | Improved bone health; |
| | | phytoestrogens | Estrogenic effect |
| 9F | 20HE + | Dioscorea napponica | Increased muscle mass; |
| | Ecdysteroids | extract; Active ingredients: | Improved bone health |
| | | dioscin | |
| 9G | 20HE + | Secosteroid hormone | Increased muscle mass; |
| | Ecdysteroids | (Vitamin D3) | Improved bone health |
| 9H | 20HE + | Citrus sinensis extract; Active | Increased muscle mass; |
| | Ecdysteroids | ingredients: Hesperidin | Improved bone health |

As shown in Table 4, the compositions disclosed herein may contain Curcuma longa extracts comprising curcumin, demethoxycurcumin, and bisdemethoxycurcumin, The Curcuma longa extracts are anti-inflamatory agents, and promote muscle regeneration after traumatic injury.

As shown in Table 5, the compositions disclosed herein may contain Ginkgo biloba extracts. where the Ginkgo biloba extracts stimulate an increase in muscle mass, and may delay sarcopenia. The compositions disclosed herein may contain Zingiber officinale extracts comprising 6-gingerol, 6-shogaol, and/or 6-paradol. The Zingiber officinale extracts are anti-inflamatory agents, and promote recovery of muscle strength following intense exercise. The compositions disclosed herein may contain Ginseng extracts, which may act to delay sarcopenia. The compositions disclosed herein may contain resveratrol in an amount effective to activate satellite cells, and/or stimulate myogenesis or hypertrophy in muscle tissue. The compositions disclosed herein may contain prickly pear cactus extracts, which contain phytoestrogens and stimulate estrogen-like activity. The compositions disclosed herein may contain an extract of Dioscorea napponica, containing dioscin as an active ingredient. The extract of Dioscorea napponica promotes osteoblast differentiation, and inhibits osteoclasts. The compositions disclosed herein may contain a secosteroid hormone, such as Vitamin D3, in an amount effective to promote muscle and bone health. The compositions disclosed herein may contain an extract of Citrus sinensis comprising hespiridin. Extracts containing hesperidin activate satellite cell activity, and promote myoblast and osteoblast differentiation.

In various embodiments, a composition of 20HE and at least one ecdysteroid may be administered as a tablet, a capsule, or an orally administered liquid formulation. The composition may be administered as a powder, or as a suspension of the active ingredients. Such a suspension may take the form of a suspension of liposomes, miceles, nanogels, cyclodextrins, dendrimers, or solid lipids having a hydrophobic core, where the active ingredients are contain in the hydrophobic core. Such suspensions provide enhanced bioavailability in plasma, and stabilize the active ingredients in plasma.

Although the various embodiments have been described in detail with particular reference to certain exemplary aspects thereof, it should be understood that the invention is capable of other embodiments and its details are capable of modifications in various obvious respects. As is readily apparent to those skilled in the art, variations and modifications can be affected while remaining within the spirit and scope of the invention. Accordingly, the foregoing disclosure, description, and figures are for illustrative purposes only and do not in any way limit the invention, which is defined only by the claims.

What is claimed is:

1. A method of treating loss of muscle mass or stimulating muscle protein synthesis in a patient in need thereof, comprising administering to said patient an effective amount of a composition comprising a combination of:
20-hydroxy-ecdysone (20HE) or a pharmaceutically acceptable salt thereof; and
at least one ecdysteroid selected from the group consisting of polypodine, makisterone A, integristerone, taxisterone, lesterone, rapisterone, inokonesterone, carthamosterone, rubrosterone, leuzeasterone, ayugasterone, turkestrone, salts thereof, and derivatives thereof.

2. The method of claim 1, wherein said composition includes an extract of at least one plant selected from the group consisting of spinach, Ajuga turkestanica, Rhaponticum carthamoides, Cyanotis longifolia, and Cyanotis arachnoidea;
wherein at least one of said 20HE and said at least one ecdysteroid is a component of said extract.

3. The method of claim 1, wherein said composition comprises said 20HE and said at least one ecdysteroid in a ratio of 1 part 20HE to between 0.01 and 100 parts of said at least one ecdysteroid.

4. The method of claim 1, wherein said composition further comprises an effective amount of hesperidin.

5. The method of claim 1, wherein said composition further comprises an effective amount of hesperidin;
wherein said composition comprises from about 200 to about 5 parts by weight of hesperidin and about one part by weight of said combination of 20HE and at least one ecdysteroid.

6. The method of claim 2, wherein said composition further comprises an effective amount of hesperidin;
wherein said composition comprises from about 200 to about 5 parts by weight of hesperidin and about one part by weight of said extract.

7. The method of claim 1, wherein said composition further comprises an effective amount of hesperidin;
wherein said composition comprises about 20 parts by weight of hesperidin and about one part by weight of said combination of 20HE and at least one ecdysteroid.

8. The method of claim 2, wherein said composition further comprises an effective amount of hesperidin;
wherein said composition comprises about 20 parts by weight of hesperidin and about one part by weight of said extract.

9. The method of claim 1, wherein said composition further comprises an effective amount of an extract of Curcuma longa, comprising curcumin, demethoxycurcumin, bis-demethoxycurcumin;
wherein said extract of Curcuma longa is effective to promote muscle regeneration.

10. The method of claim 9, wherein said composition further comprises an effective amount of an additional component selected from the group consisting of:
an extract of Piper nigrum; an extract of Cardo mariano; an extract of Allium cepa; silibinin; and quercetin;
wherein said additional component is effective to inhibit curcumin glucuronidation.

11. The method of claim 1, wherein said composition further comprises an effective amount of an additional component selected from the group consisting of:
an extract of Ginkgo biloba in an amount effective to increase muscle mass, or delay sarcopenia;
an extract of Zingiber officinale comprising 6-gingerol, 6-shogaol, 6-paradol, or a mixture thereof, said extract of Zingiber officinale being present in an amount effective to promote recovery of muscle strength following exercise;
an extract of ginseng in an amount effective to delay sarcopenia;
resveratrol in an amount effective to stimulate at least one of i) activation of satellite cells, ii) myogenesis in myscle tissue; and iii) hypertrophy in muscle tissue;
an extract of prickly pear cactus comprising at least one phyto-estrogen;
an extract of Dioscorea napponica comprising dioscin, said extract of Dioscorea napponica being present in an amount effective to promote at least one of osteoblast differentiation and osteoclast inhibition;
a secosteroid hormone; and
an extract of Citrus sinensis in an amount effective to stimulate at least one of i) activation of satellite cells, ii) myoblast differentiation; and iii) osteoblast differentiation.

12. The method of claim 1, wherein said combination of 20HE and at least one ecdysteroid is a component of an extract of at least one plant selected from the group consisting of spinach, Ajuga turkestanica, Rhaponticum carthamoides, Cyanotis longifolia, and Cyanotis arachnoidea.

13. The method of claim 12, wherein said composition further comprises an effective amount of an extract of Curcuma longa, comprising curcumin, demethoxycurcumin, bis-demethoxycurcumin;
wherein said extract of Curcuma longa is effective to promote muscle regeneration.

14. The method of claim 13, wherein said composition further comprises an effective amount of an additional component selected from the group consisting of:
an extract of Piper nigrum; an extract of Cardo mariano; an extract of Allium cepa; silibinin; and quercetin;
wherein said additional component is effective to inhibit curcumin glucuronidation.

15. The method of claim 12, wherein said composition further comprises an effective amount of an additional component selected from the group consisting of:
an extract of Ginkgo biloba in an amount effective to increase muscle mass, or delay sarcopenia;
an extract of Zingiber officinale comprising 6-gingerol, 6-shogaol, 6-paradol, or a mixture thereof, said extract of Zingiber officinale being present in an amount effective to promote recovery of muscle strength following exercise;
an extract of ginseng in an amount effective to delay sarcopenia;
resveratrol in an amount effective to stimulate at least one of i) activation of satellite cells, ii) myogenesis in myscle tissue; and iii) hypertrophy in muscle tissue;
an extract of prickly pear cactus comprising at least one phyto-estrogen;
an extract of Dioscorea napponica comprising dioscin, said extract of Dioscorea napponica being present in an amount effective to promote at least one of osteoblast differentiation and osteoclast inhibition;
a secosteroid hormone;
an extract of Citrus sinensis in an amount effective to stimulate at least one of i) activation of satellite cells, ii) myoblast differentiation; and iii) osteoblast differentiation:

16. A composition for treating loss of muscle mass or stimulating muscle protein synthesis, comprising an effective amount of:
a) a composition comprising a combination of:
20-hydroxy-ecdysone (20HE) or a pharmaceutically acceptable salt thereof; and
at least one ecdysteroid selected from the group consisting of polypodine, makisterone A, integristerone, taxisterone, lesterone, rapisterone, inokonesterone, carthamosterone, rubrosterone, leuzeasterone, ayugasterone, turkestrone, salts thereof, and derivatives thereof; and optionally
b) hesperidin.

17. The composition of claim 16, wherein said composition comprises from about 200 parts by weight to about 5 parts by weight of hesperidin and about one part by weight of said combination of 20HE and at least one ecdysteroid.

18. The composition of claim 16, wherein said composition further comprises an effective amount of an extract of Curcuma longa, comprising curcumin, demethoxycurcumin, bis-demethoxycurcumin;
wherein said extract of Curcuma longa is effective to promote muscle regeneration.

19. The composition of claim 18, wherein said composition further comprises an effective amount of an additional component selected from the group consisting of:
an extract of Piper nigrum; an extract of Cardo mariano; an extract of Allium cepa; silibinin; and quercetin;
wherein said additional component is effective to inhibit curcumin glucuronidation.

20. The composition of claim 16, wherein said composition further comprises an effective amount of an additional component selected from the group consisting of:
an extract of Ginkgo biloba in an amount effective to increase muscle mass, or delay sarcopenia;
an extract of Zingiber officinale comprising 6-gingerol, 6-shogaol, 6-paradol, or a mixture thereof, said extract of Zingiber officinale being present in an amount effective to promote recovery of muscle strength following exercise;
an extract of ginseng in an amount effective to delay sarcopenia;
resveratrol in an amount effective to stimulate at least one of i) activation of satellite cells, ii) myogenesis in myscle tissue; and iii) hypertrophy in muscle tissue;
an extract of prickly pear cactus comprising at least one phyto-estrogen;
an extract of Dioscorea napponica comprising dioscin, said extract of Dioscorea napponica being present in an amount effective to promote at least one of osteoblast differentiation and osteoclast inhibition;
a secosteroid hormone;
an extract of Citrus sinensis in an amount effective to stimulate at least one of i) activation of satellite cells, ii) myoblast differentiation; and iii) osteoblast differentiation:
